(19) United States
(12) Patent Application Publication
(10) Pub. No.: US 2025/0213815 A1
(43) Pub. Date: Jul. 3, 2025

(54) Infant Calming Device

(71) Applicant: Oliver Reisenweber, McKinney, TX (US)

(72) Inventor: Oliver Reisenweber, McKinney, TX (US)

(21) Appl. No.: 18/960,294

(22) Filed: Nov. 26, 2024

Related U.S. Application Data
(60) Provisional application No. 63/616,156, filed on Dec. 29, 2023.

(51) Int. Cl. A61M 21/02

(52) U.S. Cl. CPC A61M 21/02; A61M 2240/00

(57) ABSTRACT

An exemplary device in accordance with the disclosed principles comprises a hollow housing formed of a rigid material. In the housing is a plurality of movable components that have a shape and composition that results in the generation of both sounds and vibrations when the components rattle around within the housing. The exemplary device further includes a motor mounted to the housing. The shaft of the motor includes an off-center weight such that rotation of the shaft causes a shaking of the motor, which, in turn, causes shaking of the housing by virtue of the motor's attachment. As the housing is shaken, the plurality of movable components are shaken around within the housing. The impact of these movable components with the interior of the housing, as well as collision of the components among one another, causes both sounds and vibrations to emanate from housing, calming a nearby infant.

### RELATED APPLICATIONS

This disclosure claims priority to U.S. Provisional Patent Application Ser. No. 63/616, 156, filed Dec. 29, 2023, which is incorporated herein by reference in its entirety for all purposes.

### TECHNICAL FIELD

The disclosed principles provide a device designed and configured to calm crying infants. Generally speaking, a device and system in accordance with the disclosed principles does so by generating a combination of vibrations and a rattling noise. In exemplary embodiments, the vibrations are caused by the rattling of a plurality of components housed within a body of the device or system. Embodiments of a disclosed device are discussed in further detail below.

### SUMMARY

In one embodiment, a device in accordance with the disclosed principles comprises a housing formed of a rigid material, such as plastic, metal or wood. The housing can take any shape that can comprise a hollow housing. Stored within the housing is a plurality of movable components, wherein the movable components have a shape and composition that results in the generation of both sounds and vibrations when the components rattle around within the housing. The exemplary device further includes a motor mounted to the housing, or alternatively mounted to a secondary housing held within the housing, and a power source providing power to the operate the motor. The shaft of the motor includes an off-center weight such that when the motor is operated to rotate the shaft, rotation of the shaft causes a shaking of the motor. The shaking of the motor, in turn, causes shaking of the housing or container within the housing by virtue of the motor's attachment to the housing. As the housing is shaken by the motor, the plurality of movable components stored within the housing are shaken around within the housing. The impact of these movable components with the interior of the housing, as well as collision of the components among one another, causes both sounds and vibrations to emanate from housing. A power regulation device, such as a potentiometer, variable resistor, or rheostat, may also be included in exemplary embodiments. Such a power regulation device may control the rotational speed of the motor, thereby varying the volume and frequency of the vibrations caused by the movement of the movable components.

### DETAILED DESCRIPTION

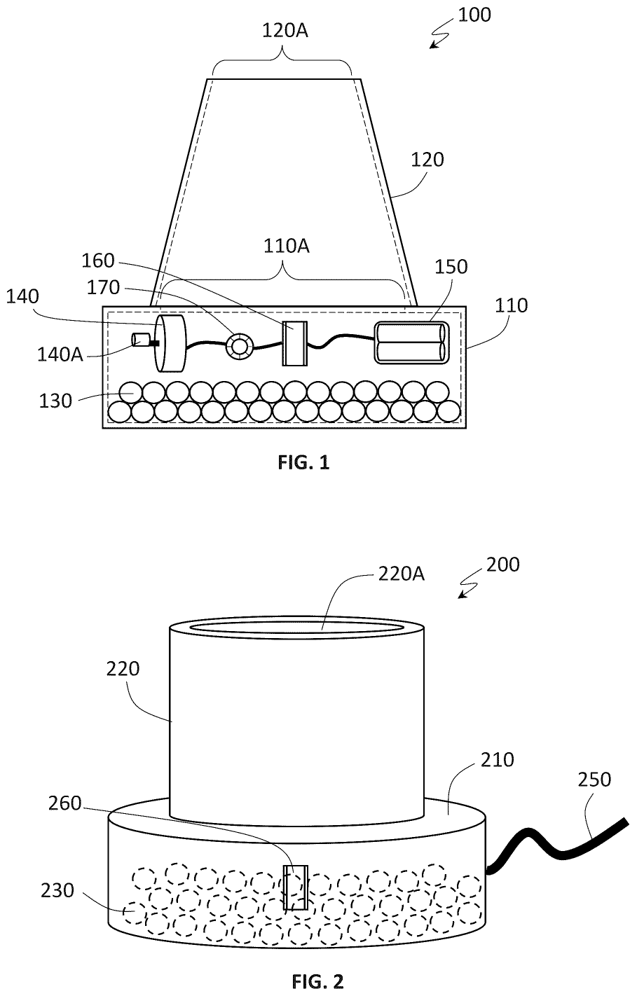

Looking more specifically at FIG. 1, illustrated is a plan view of one embodiments of an infant calming device 100 designed and constructed in accordance with the disclosed principles. This embodiment of a disclosed device includes a housing 110 with an acoustic magnifier 120 coupled to a top surface of the housing. In this exemplary embodiment, the housing comprises rigid plastic, but in other embodiments, the housing may be formed of any rigid material, such as plastic, metal, or wood, capable of providing an acoustic and vibrational response to the impact of a plurality of movable components 130 housed within the housing 110, which is described in further detail below.

Stored within the housing 110 is a plurality of movable components 130. These movable components 130 are housed loosely within the interior of the housing 110 such that they can move around freely within the housing 110 and with respect to one another. Such movement of the movable components 130 within the housing 110 results in the generation of both acoustic waves and vibrations as a result of the movable components 130 impacting the interior surfaces of the housing 110. To create the shaking and other movement of the movable components 130 within the housing 110, a motor 140 is mounted to the housing 110, for example, to an interior wall of the housing 110. In other embodiments, the motor 140 may actually be mounted to a container or secondary housing, which in turn is mounted to the housing 110 of the device 100. In such embodiments, the motor 140 is configured to shake the secondary housing, which in turn causes the primary housing 110 to shake. In yet other embodiments, the motor 140 may be mounted in such an internal secondary housing or similar container, and that secondary housing may simply be placed within the housing 110. In such embodiments, the motor 140 is configured to shake the secondary housing, which in turn moves among the movable components 130 within the primary housing 110 to cause the sounds and vibrations described herein.

In exemplary embodiments, the motor 140 is an electric motor, which is powered by an electrical power supply 150. The power supply 150 may comprise batteries, disposable or rechargeable, such that the device 100 is portable or otherwise untethered, or may comprise a corded electrical power source such as is shown in FIG. 2 and discussed below. To activate and deactivate operation of the electric motor 140, a power switch 160 may be provided between the motor 140 and the power supply 150. Additionally, a power regulation device 170 may be provided to regulate power from the power supply 150 to the motor 140. For example, the power regulation device 170 may comprise a potentiometer, also known as a variable resistor or rheostat. While the illustrated embodiment shows the power regulation device 170 separate from, and electrically coupled to, the power switch 160, in some embodiments the power regulation device 170 may be comprised within the power switch 160.

Once activated by the switch 160, the motor 140 spins its rotor, which then spins the shaft extending from the motor's body. Mounted on the shaft of the motor 140 is an off-center weight 140A rather than a pinion gear or other mechanism. The off-center weight 140A resembles am offset cam such that when the motor 140 is operated to rotate its shaft, rotation of the off-center weight 140A causes an intentional shaking of the motor 140. Because the motor 140 is mounted to the housing 110, the shaking of the motor 140 causes shaking of the housing 110. In turn, shaking of the housing 110 causes shaking of the plurality of movable components 130 held within the housing 110 to generate sounds and vibrations by impacting the interior of the housing 110. The power regulation device 170, if included, may then be operated to adjust the rotational speed of the motor 140 during use of the device 100. As the speed of the motor 140 is adjusted, both the frequency and volume of the acoustic waves and vibrations emanating from the device 100 can be increased or decreased, as desired.

The housing 110 can take any shape, such as the illustrated three-dimensional rectilinear shape like a cube or similar structure. However, in other embodiments, the housing may have the shape of a cylinder, a cone, a sphere, or any other shape that can comprise a hollow housing. The acoustic magnifier 120 is positioned on a top surface of the housing 110, and in advantageous embodiments comprises a hollow shape like a megaphone. The top surface of the housing 110 may include an opening 110A such that sound and vibrational waves may exit the housing 110 and travel into the acoustic magnifier 120 with no resistance other than the ambient air. In this illustrated embodiments, the acoustic magnifier 120 comprises a truncated cone shape that tapers inward when extending from the housing 110, but in other embodiments the truncated cone shape may taper outwardly when extending from the housing 110. The acoustic magnifier 120 may also be comprised of a rigid material, and in some embodiments may even comprise the same material as the housing 110. In some embodiments, the housing 110 and acoustic magnifier 120 may be integrally formed, while in other embodiments, the acoustic magnifier 120 may be attached to the housing, such as with an adhesive, fasteners, heat welding, or any other advantageous means of coupling the acoustic magnifier 120 to the housing 110.

As the acoustic waves and vibrations are generated, many of the waves and vibrations escape from the housing 110 through the path of least resistance, which is the opening 110A at the top of the housing 110. As these acoustic waves and vibrations exit the housing 110, they are funneled into the acoustic magnifier 120, which is also hollow in design. As such, these acoustic waves and vibrations travel up the acoustic magnifier 120 such that they are acoustically focused at the upper exit 120A of the acoustic magnifier 120. As a result, while some acoustic waves and vibrations emanate from the sidewalls of the housing 110 as the movable components 130 shake and rattle therein, the acoustic waves and vibrations exiting the acoustic magnifier 120 are amplified by being focused together within the acoustic magnifier 120.

In some embodiments, the movable components 130 comprise spherically-shaped components, while in other embodiments the movable components 130 comprise rectilinearly-shaped components, such as cubes. Other shapes, whether symmetrical or non-symmetrical, or uniform or non-uniform, for the movable components 130 may also be employed. The composition of the movable components 130 may be wood, metal, plastic, glass, or any other rigid material capable of producing acoustic waves and vibrations when shaken within the housing 110. Moreover, a combination of movable components 130 constructed from varying materials may also be employed. Still further, the one or more materials comprising the movable components 130 may be selected, as well as the rigidity of each such material, based on the material or materials comprising the housing 110. Such selection of materials permits altering the pitch of the acoustic waves emanating from the device 100 when in use. A muffling or pitch-altering component (not illustrated) may also be included on the device 100. Such a component may be used on the acoustic magnifier 120, on the housing 110, or on both, to alter/adjust the pitch of the acoustic waves and vibrations emitting from the device 100 during its use.

Furthermore, in some embodiments, the device 100 may comprise a timing device (not illustrated), either within the activation switch 160 or electrically coupled to it, which may be used to power on and/or power off the device 100 via a timing function. In such embodiments, the device 100 may be set to turn off after a predetermined amount of time has expired on the timing device. Also, any of various remote activation devices (not illustrated) may be included in or coupled to the power switch 160. For example, a sound-activated device may be included with the device 100 such that a noise at a predetermined level, such as an infant's cry, may be used to activate the power switch 160. In related embodiments, the sound-activated device may itself include a variable adjustment device such that the sensitivity of the sound-activated device can be adjusted to a desired level at which the device is activated. In still further embodiments, a combination of a sound-activated device and a timing device may be used so that the device 100 is activated by a predetermined level of sound, but then deactivated once a predetermined time interval has expired.

Experiments with various embodiments of a device designed and constructed in accordance with the disclosed principles by the inventor have proven successful in calming an infant who is fussy or crying. Specifically, the acoustic noise and vibrations emanating from the device 100 during use in proximity to an infant were shown to draw the attention of the infant, and thereby calm the infant's disposition. It is believed that the vibrations generated by a device as disclosed herein are part of the success of the device in calming infants.

Turning now to FIG. 2, illustrated is an isometric view of another embodiment of an infant calming device 200 designed and constructed in accordance with the disclosed principles. This embodiment of a disclosed device 200 again includes a housing 210 and an acoustic magnifier 220 positioned on the top surface of the housing 210. As before, the top surface of the housing 210 includes an opening (not illustrated) that permits acoustic waves and vibrations generated from within the housing 210 to escape upward into the acoustic magnifier 220. An opening 220A at the top of the acoustic magnifier 220 permits magnified acoustic waves and vibrations to emanate from the top of the acoustic magnifier 220. The housing 210 and the acoustic magnifier 220 may be constructed of any rigid material capable of generating acoustic waves and vibrations from the shaking of the movable components 230 held within the housing 210.

The movable components 230 may again be comprised of any one or more advantageous rigid material(s), and such material(s) may be selected so as to promote not only the generation of acoustic waves and vibrations from the device 200, but also the pitch of those acoustic waves. Also as before, a power switch 260 may be included to activate and deactivate the motor (not illustrated) within the device 200, as well as a power regulation unit (not illustrated) to regulate the speed of the motor. As described above, regulating the speed of the motor can be used to alter the pitch and frequency of the acoustic waves and vibrations generated by the device 200. The embodiment illustrated in FIG. 2 differs from the embodiment of FIG. 1 in that the device 200 in FIG. 2 includes a hardwired power cord 250 to plug in the device 200 to an electrical power source. Alternatively, the power cord 250 may instead be embodied as a charging cable for a rechargeable power source (not illustrated) located within the device 200. Also different in the device 200 of FIG. 2 is the shape of both the housing 210 and acoustic magnifier 220. More specifically, both of these component as cylindrical in shape, and the acoustic magnifier 220 is not tapered as in the embodiment of FIG. 1. These differences in shapes between the embodiments in FIGS. 1 and 2 are illustrated to demonstrate that many available shapes may be used to form the housing 210 and/or acoustic magnifier 220, without departing from the spirit and scope of the disclosed principles.

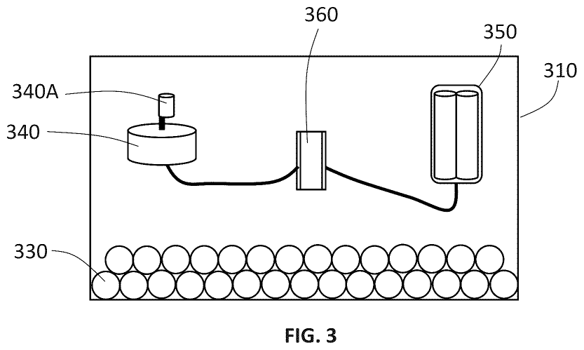

Turning now to FIG. 3, illustrated is a plan view of a third embodiment of an infant calming device 300 designed and constructed in accordance with the disclosed principles. This embodiment of a disclosed device 300 again includes a housing 310, but does not include an acoustic magnifier on the top surface of the housing 310. As such, this embodiment of a device 300 as disclosed herein is more compact embodiment of the device 300. Such a compact design may prove advantageous for use in travel situations, where size of the device 300 is more important than at home.

During operation of this embodiment of the device 300, acoustic waves and vibrations generated by the shaking and impacts of the movable components 330 within the housing 310 emanate from the walls and top surface of the housing 310. As before, activation and deactivation of the device 300 may be accomplished by a power switch 360, which provides or removes, respectively, power from the motor 340. With the off-center component 340A on the shaft of the motor 340, rotation of the motor 340 again results in shaking of the housing 310 of the device 300, which in turn results in shaking of the movable components 330 within the housing 310. Optionally, a power regulation unit (not illustrated) may be included to regulate the rotational speed of the motor 340 to thereby alter the frequency and volume of the acoustic waves and vibrations generated by the shaking of the movable components 330 within the housing 310 as before. Also as before, the materials used for construction of the housing 310 and the movable components 330 may be selected to achieve a desired pitch of the acoustic waves generated by the device 300. The housing 310 may also include one or more liners on the interior of the housing 310 selected in both material(s) and thickness(es) to affect the sounds and/or vibrations emanating from the device 300. Moreover, such liner(s) or lining(s) may instead or additionally be added to the exterior of the housing 310, again specifically selected to affect the sounds and/or vibrations emanating from the device 300.

In some embodiments, a laminate (i.e., layered) material may be employed for the housing or other components of a device as disclosed herein. Such laminate material may be a composite of multiple layers of the same type of material (e.g., various different wood materials, plastic materials, metal materials, etc.), or may be a composite of multiple layers of different materials. Additionally, such laminate composite of different materials may be selected such that a certain material is on the interior of the housing, and other material(s) selected to be on the exterior of the housing or the inner layer(s) of the housing laminate material. For example, the laminate material may be made with a wood layer on the interior of the housing and a plastic or metal layers on the exterior, such as to enhance the exterior strength and/or appearance of the device. In other embodiments, the laminate material may be made with a metal or plastic layer on the interior of the housing, such as to result in louder or certain desired acoustic waves generated from the interior components impacting the metal or plastic interior layer, and then a wood or other material with dampening properties (as compared to metal or plastic) to provide not only an aesthetic appearance of the housing, but also to somewhat dampen the acoustic waves generated with the metal or plastic interior layer. This could be the case if the desired frequency of acoustic waves is only generated by the selected plastic or metal interior layer, but the volume/amplitude of those generated waves is too loud.

Still further, a sleeve comprised of one or more materials (and/or one or more parts) may also be provided to surround the exterior of the housing in order to offer such a dampening effect. Moreover, such a sleeve may be removable or adjustable, such as being slidable (or otherwise movable with respect to the housing) to cover some or all of the exterior of the housing, as desired, to intentionally affect the frequency and/or amplitude of the acoustic waves generated from the housing. Such a sleeve may also be decorative, and may be comprises of a single material or again as a laminate material with the various layers, and the order (i.e., from interior to exterior) selected to affect the generated acoustic waves in the desired manner. Of course, a combination of any of these select embodiments may be chosen where the various selection can advantageously operate together to have the desired effect on the generated acoustic waves.

While this disclosure has been particularly shown and described with reference to preferred embodiments, it will be understood by those skilled in the pertinent field art that various changes in form and detail may be made therein without departing from the spirit and scope of the invention. The inventor expects skilled artisans to employ such variations as appropriate, and the inventors intend the invention to be practiced otherwise than as specifically described herein. Accordingly, this disclosure includes all modifications and equivalents of the subject matter recited in the claims appended hereto, as permitted by applicable law. Moreover, any combination of the above-described elements in all possible variations thereof is encompassed by the disclosure unless otherwise indicated herein or otherwise clearly contradicted by context.

Also, while various embodiments in accordance with the principles disclosed herein have been described above, it should be understood that they have been presented by way of example only, and not limitation. Thus, the breadth and scope of this disclosure should not be limited by any of the above-described exemplary embodiments, but should be defined only in accordance with any claims and their equivalents issuing from this disclosure. Furthermore, the above advantages and features are provided in described embodiments, but shall not limit the application of such issued claims to processes and structures accomplishing any or all of the above advantages.

The uses of the terms “a” and “an” and “the” and similar references in the context of describing the invention(s) (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically, and by way of example, although the headings refer to a “Technical Field,” the claims should not be limited by the language chosen under this heading to describe the so-called field. Further, a description of a technology as background information is not to be construed as an admission that certain technology is prior art to any embodiment(s) in this disclosure. Neither is the “Summary” to be considered as a characterization of the embodiment(s) set forth in issued claims. Furthermore, any reference in this disclosure to “invention” in the singular should not be used to argue that there is only a single point of novelty in this disclosure. Multiple embodiments may be set forth according to the limitations of the multiple claims issuing from this disclosure, and such claims accordingly define the embodiment(s), and their equivalents, that are protected thereby. In all instances, the scope of such claims shall be considered on their own merits in light of this disclosure, but should not be constrained by the headings set forth herein.

Moreover, the Abstract is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the claims reflect, inventive subject matter lies in less than all features of a single disclosed embodiment. Thus, the following claims are hereby incorporated into the Detailed Description, with each claim standing on its own as a separate embodiment.

Any and all publications, patents, and patent applications cited in this specification are herein incorporated by reference as if each individual publication, patent, or patent application were specifically and individually indicated to be incorporated by reference and set forth in its entirety herein.

What is claimed is:

1. A device, comprising:
a housing formed of a rigid material;
a plurality of movable components stored within the housing;
a motor in contact with the housing and having an off-center weight mounted on the its shaft, wherein rotation of the shaft causes a shaking of the motor;
a power source providing power to the motor for rotation of the shaft;
wherein the shaking of the motor causes shaking of one or both of the housing and plurality of movable components, and wherein the shaking of the housing causes shaking of the plurality of components therein to generate sounds and vibrations by impacting the housing.

2. The device according to claim 1, wherein the movable components comprise spherically-shaped components.

3. The device according to claim 1, wherein the movable components comprise rectilinearly-shaped components.

4. The device according to claim 1, wherein the movable components comprise wood.

5. The device according to claim 1, wherein the movable components comprise metal.

6. The device according to claim 1, wherein the housing comprises plastic.

7. The device according to claim 1, wherein the housing comprises metal.

8. The device according to claim 1, wherein the housing further comprises an opening, the device further comprising an acoustic magnifier coupled to the opening for magnifying sound emanating from the housing.

9. The device according to claim 8, wherein the acoustic magnifier comprises the same material as the housing.

10. The device according to claim 1, further comprising a power regulation device regulating rotational speed of the motor, wherein rotational speed of the motor is directly proportional to the acoustic waves and vibrations generated by the device.

11. A method of manufacturing an infant calming device, the method comprising:
forming a housing formed of a rigid material;
storing a plurality of movable components within the housing;
mounting a motor in contact with the housing, the motor having an off-center weight on the its shaft wherein rotation of the shaft causes a shaking of the motor;
providing power via a power source to the motor for rotation of the shaft;
wherein the shaking of the motor causes shaking of one or both of the housing and plurality of movable components, and wherein the shaking of the housing causes shaking of the plurality of components therein to generate sounds and vibrations by impacting the housing.

12. The method according to claim 11, wherein the movable components comprise spherically-shaped components.

13. The method according to claim 11, wherein the movable components comprise rectilinearly-shaped components.

14. The method according to claim 11, wherein the movable components comprise wood.

15. The method according to claim 11, wherein the movable components comprise metal.

16. The method according to claim 11, wherein the housing comprises plastic.

17. The method according to claim 11, wherein the housing comprises metal.

18. The method according to claim 11, further comprising:
forming an opening in the housing; and
coupling an acoustic magnifier to the opening for magnifying sound emanating from the housing.

19. The method according to claim 18, wherein the acoustic magnifier comprises the same material as the housing.

20. The method according to claim 11, further comprising regulating rotational speed of the motor with a power regulation device, wherein rotational speed of the motor is directly proportional to the acoustic waves and vibrations generated by the device.
